(19) United States
(12) Patent Application Publication
(10) Pub. No.: US 2017/0304002 A1
(43) Pub. Date: Oct. 26, 2017

(54) ELECTROMANIPULATION OF PROTEINS USING NANOSECOND PULSED ELECTRIC FIELDS

(71) Applicant: Old Dominion University, Norfolk, VA (US)

(72) Inventors: Stephen J. BEEBE, Norfolk, VA (US); Karl H. SCHOENBACH, Norfolk, VA (US)

(21) Appl. No.: 15/507,820

(22) PCT Filed: Sep. 2, 2015
(86) PCT No.: PCT/US15/48209

§ 371 Date: Mar. 1, 2017

Related U.S. Application Data
(60) Provisional application No. 62/044,613, filed on Sep. 2, 2014.

(51) Int. Cl. A61B 18/20; C12N 15/70; C12N 15/10; A61B 18/18; C12N 5/00; A61B 18/00

(52) U.S. Cl. CPC A61B 18/20; C12N 15/101; A61B 18/18; C12N 15/70; A61B 2018/00994; C12N 5/00; C12N 2529/00

(57) ABSTRACT

The present disclosure describes methods for intracellular electromanipulation of proteins using nanosecond pulsed electric fields (nsPEFs). The nsPEFs have effects on proteins in addition to permeabilizing cellular membranes. The nsPEFs induce a Ca²⁺-dependent dissipation of the mitochondria membrane potential (ΔΨm), which is enhanced when high frequency components are present in fast rise-fall waveforms. Ca²⁺ is shown to have little or no effect on propidium iodide uptake as a measure of plasma membrane poration and consequently intracellular membranes. Since Ca²⁺-regulated events are mediated by proteins, actions of nsPEFs on proteins that regulate and/or affect the mitochondria membrane potential are possible. Given that nsPEF-induced dissipation of ΔΨm was more effective when high frequency components were present in fast rise time waveforms, the effects on proteins are due to these high frequency components. These results present direct evidence that nsPEFs affect proteins and their functions by affecting their structure.

### CROSS-REFERENCE TO RELATED APPLICATIONS

This application claims priority to U.S. Provisional Application Ser. No. 62/044,613, filed Sep. 2, 2014, which is hereby incorporated by reference.

### BACKGROUND

### Field of the Disclosure

The present disclosure relates generally to electrotherapy, and more specifically, to methods for intracellular electromanipulation of proteins using nanosecond pulsed electric fields (nsPEFs).

### Background Information

Electric fields can be used to manipulate cell function in a variety of ways. Some specific cell structures that can be affected by electric fields are the lipid bilayer of plasma membranes and effects on intracellular membranes that extend from the plasma membrane. Effects of conventional electroporation pulses with relatively long pulse durations in the microsecond (μs) and millisecond (ms) ranges and relative low electric fields (up to 1 kV/cm) have mostly focused exclusively on the lipid bilayer of plasma membranes. While electroporation pulses may have some effects on intracellular membranes that extend from the plasma membrane (see A. T. Esser, K. C. Smith, T. R. Gowrishankar, Z. Vasilkoski, J. C. Weaver, Mechanisms for the intracellular manipulation of organelles by conventional electroporation, Biophys. J. 98 (2010) 2506-2514), the general focus has remained on plasma membranes.

More recently, attention has shifted to employment of pulses in the nanosecond range including relative high electric fields (tens of kV/cm), so called nanosecond pulsed electric fields (nsPEFs). Considerations for nsPEF effects are not only on plasma membranes, but also on intracellular membranes of vesicles, endoplasmic reticulum (ER), mitochondria, nucleus and other organelles. While biological membranes are decorated with integral and peripheral proteins, both modeling and experimental approaches with electric fields have fixated on lipid structures. If arrays of proteins embedded in plasma membranes are silent, non-responders to electric fields, analysis of lipid bilayer structures may provide the fundamental understanding of bioelectric effects on cell structures and functions. Indeed, much has been learned by analyzing electric field effects on plasma membrane lipids and there is considerable congruence with experimental and modeling data on many fronts. However, there are a number of observations that are not consistent with or explained by actions of electric fields on plasma membranes.

By using pulses in the sub-microsecond range, pulsed power devices with high voltage capacitors and fast discharge capabilities compress electric energy and release it in nanosecond (ns) or picosecond (ps) instances, thereby greatly increasing the power released into cells or tissues. These nsPEFs are high power, low energy, non-thermal pulses. The conventional understanding is that nsPEFs have a unique capacity to impact intracellular structures, such as cell organelles. This was initially hypothesized and demonstrated by breaching vesicular membranes in human eosinophils (see K. H. Schoenbach, S. J. Beebe, E. S. Buescher, Intracellular effect of ultrashort electrical pulses, Bioelectromagnetics 22 (2001) 440-448). Distinct effects of nsPEFs have also been observed to rapidly and transiently release Ca²⁺ from intracellular stores (see: P. T. Vernier, Y. Sun, L. Marcu, G. Salemi, C. M. Craft, M. A. Gundersen, Ca2+ bursts induced by nanosecond electric pulses, Biochem. Biophys. Res. Commun. 310 (2003) 286-295; J. A. White, P. F. Blackmore, K. H. Schoenbach, S. J. Beebe, Stimulation of capacitive Ca2+ entry in HL-60 cells by nanosecond pulsed electric fields, J. Biol. Chem. 279 (2004) 22964-22972; E. S. Buescher, R. R. Smith, K. H. Schoenbach, Submicrosecond intense pulsed electric field effects on intracellular free Ca²⁺: mechanisms and effects, IEEE Trans. Plasma Sci. 32 (2004) 1563-1572; I. Semenov, S. Xiao, O. N. Pakhomova, A. G. Pakhomov, Recruitment of the intracellular Ca²⁺ by ultrashort electric stimuli: the impact of pulse duration, Cell Calcium54 (2013) 145-150; and I. Semenov, S. Xiao, A. G. Pakhomov, Primary pathways of intracellular Ca(2+) mobilization by nanosecond pulsed electric field, Biochim. Biophys. Acta 2013 (1828) 981-989).

This Ca²⁺ release is believed to be due to permeabilization of the ER with Ca²⁺ diffusing down its electrochemical gradient into the cytosol. This intracellular release of Ca²⁺ has most closely been associated with Ca²⁺-mediated intracellular signaling and has been demonstrated to activate platelets (see J. Zhang, P. F. Blackmore, B. Y. Hargrave, S. Xiao, S. J. Beebe, K. H. Schoenbach, Nanosecond pulse electric field (nanopulse): a novel non-ligand agonist for platelet activation, Arch. Biochem. Biophys. 471 (2008) 240-248), and modular contractility of cardiomyocytes (see S. Wang, J. Chen, M. T. Chen, P. T. Vernier, M. A. Gundersen, M. Valderrabano, Cardiac myocyte excitation by ultrashort high-field pulses, Biophys. J. 96 (2009) 1640-1648). Since proteins embedded on/within intracellular membranes play an important role in many cellular functions, there is a need to further examine the effects of electric fields on proteins as well as explore ways to manipulate their functions and structures for therapeutic purposes.

### SUMMARY

One or more aspects of the present disclosure provide methods for intracellular electromanipulation of proteins using nanosecond pulsed electric fields (nsPEFs). The method comprises applying at least one nsPEF to one or more cells, whereby proteins are embedded in plasma membranes. The at least one nsPEF has a pulse duration of at least about 60 nanoseconds and no more than about 600 nanoseconds and an electric field strength from about 0 kV/cm to about 60 kV/cm.

In one or more embodiments of the disclosure, at least one nsPEF is applied to the cells. The cells may be suspended in a medium or present as part of a tissue. The cells may be any prokaryotic or any eukaryotic cells, including but not limited to fat cells, bone cells, vascular cells, muscle cells, cartilage cells, stem cells or a combination thereof. The cells may also be abnormal cells, including cancer cells.

In some embodiments, the nsPEFs induce a Ca²⁺-dependent dissipation of the mitochondria membrane potential (ΔΨm), which is enhanced when high frequency components are present in fast rise-fall waveforms. Ca²⁺ is shown to have little or no effect on propidium iodide uptake as a measure of plasma membrane poration and consequently intracellular membranes. Since Ca²⁺-regulated events are mediated by proteins, actions of nsPEFs on proteins that regulate and/or affect the mitochondria membrane potential are possible. Given that nsPEF-induced dissipation of ΔΨm was more effective when high frequency components were present in fast rise time waveforms, it is possible that effects on proteins are due to these high frequency components. These results present direct evidences that nsPEFs affect proteins and their functions by affecting their structure.

In some embodiments, nsPEFs inactivated the C-subunit of PKA, which is the prototype of the protein kinase super family that share a common catalytic mechanism and whose functions are highly dependent on their structure, and exhibit highly conserved catalytic mechanisms. As observed for other nsPEF effects, this inactivation is independent in energy density and more related to a charging effect defined by the formula Eτn^{0.5}. Given that nsPEF-induced dissipation of ΔΨm was more effective when high frequency components were present in fast rise time waveforms, it is possible that effects on proteins are due to these high frequency components.

### BRIEF DESCRIPTION OF THE DRAWINGS

FIG. 1 is a graphical representation illustrating effects of EGTA and EGTA/BAPTA on nanosecond pulsed electric field (nsPEF)-induced dissipation of mitochondria membrane potential (ΔΨm) in Jurkat A3 cells, according to an embodiment.

FIG. 2 is a graphical representation illustrating effects of Ca²⁺ on nsPEF-induced permeabilization of plasma membranes, according to an embodiment.

FIG. 3 is a graphical representation illustrating effects of inhibitors of the mitochondria permeability transition pore (mPTP) complex on nsPEF-induced loss of ΔΨm, according to an embodiment.

FIG. 4 is a graphical representation illustrating effects of nsPEFs on enzyme activity of the catalytic subunit of the cAMP-dependent protein kinase (PKA), according to an embodiment.

### DETAILED DESCRIPTION

The present invention is described with reference to the attached figures. The figures are not drawn to scale and they are provided merely to illustrate the instant invention. Several aspects of the invention are described below with reference to example applications for illustration. It should be understood that numerous specific details, relationships, and methods are set forth to provide a full understanding of the disclosure. One having ordinary skill in the relevant art, however, will readily recognize that the embodiments of the disclosure can be practiced without one or more of the specific details or with other methods. In other instances, well-known structures or operations are not shown in detail to avoid obscuring the aspects of the disclosure. The present disclosure is not limited by the illustrated ordering of acts or events, as some acts may occur in different orders and/or concurrently with other acts or events. Furthermore, not all illustrated acts or events are required to implement a methodology in accordance with the present disclosure.

### Definitions

As used here, the following terms have the following definitions:

“Electroporation or electroporated” refers to a physical method that uses an electrical pulse to create temporary pores in cell membranes, thereby inducing necrosis or apoptosis on the electroporated cells.

“Mitochondrial membrane potential (ΔΨm)” refers to a parameter of mitochondrial function that acts as an indicator that the cells will be able to convert oxygen to cellular energy.

“Nanosecond pulsed electric fields (nsPEFs)” refers to electric pulses in the nanosecond range (about 100 picoseconds to about 1 microsecond) with electric field intensities from about 0 kV/cm to about 350 kV/cm.

### DESCRIPTION OF THE DISCLOSURE

For the purposes of promoting an understanding of the principles of the disclosure, reference will now be made to preferred embodiments and specific language will be used to describe the same. It will nevertheless be understood that no limitation of the scope of the disclosure is thereby intended. Rather, such alterations and further modifications of the disclosure, and such further applications of the principles of the disclosure as illustrated herein, as would be contemplated by one having skill in the art to which the disclosure relates are intended to be part of the present disclosure.

For example, features illustrated or described as part of one embodiment can be used on other embodiments to yield a still further embodiment. Additionally, certain features may be interchanged with similar devices or features not mentioned yet which perform the same or similar functions. It is therefore intended that such modifications and variations are included within the totality of the present disclosure.

One or more embodiments of the present disclosure are directed to methods for intracellular electromanipulation of proteins using nanosecond pulsed electric fields (nsPEFs). The methods comprise applying at least one nsPEF to one or more cells, whereby proteins are embedded in plasma membranes. The at least one nsPEF has a pulse duration of at least about 60 nanoseconds and no more than about 600 nanoseconds and an electric field strength from about 0 kV/cm to about 60 kV/cm.

The methods for intracellular electromanipulation described herein may be used for a variety of intracellular protein types. In an example, Jurkat cells are employed. In another example, the methods described herein can be used to affect structures and functions of intracellular proteins in all prokaryotic and eukaryotic cells, including but not limited to, fat cells, bone cells, vascular cells, muscle cells, cartilage cells, and stem cells. In a further example, the methods described herein can be used to affect structures and functions of intracellular proteins in abnormal cells, including cancer cells.

Reference will now be made to specific examples illustrating the use of nsPEFs for intracellular electromanipulation of proteins. It is to be understood that the examples are provided to illustrate preferred embodiments and that no limitation of the scope of the disclosure is intended thereby.

### Example 1. NsPEFs-Induced Dissipation of Mitochondria Membrane Potential (ΔΨm)

a) Materials and Methods

Cell Culture and Treatment with nsPEFs

Wildtype Jurkat T-lymphocytes (clone A3) were obtained from ATCC (Manassas, Va.) and cultured in RPMI 1640 medium (ATCC) including about 10% fetal bovine serum (FBS) (Atlanta Biologist). N1-S1 HCC cells were obtained from ATCC and cultured in Iscove's Modified Delbecco's Medium including FBS. Both cell lines were maintained in media including about 1% L-glutamine and about 1% penicillin and streptomycin. Cells were treated in cuvettes in cell culture media with nsPEFs using pulse generators, such as, for example as described in Transient features in nanosecond pulsed electric fields differentially modulate mitochondria and viability, PLoS One 7 (2012).

Flow Cytometric Analysis of ΔΨm

Loss in ΔΨm was determined by staining cells with either 2 μM JC-1 (5′,6, 6′-tetrachloro-1,1′,3,3′-tetraethylbenzimidazole-carbocyanideiodine, Molecular Probes, Eugene, Oreg., USA), where JC-1 changes from red to green when ΔΨm decreases (data not shown) or with 200 μM TMRE (tetramethylrhodamine ethyl ester, Immunochemistry Technologies LLC, Bloomington, Minn.), where TMRE red emissions decrease when ΔΨm decreases. To determine effects of Ca²⁺ on ΔΨm, cells were pre-incubated with or without 20 μM BAPTA-AM and/or 5 mM EGTA for about 30 min before nsPEF treatment. The ΔΨm was determined about 10 min after treatment with nsPEFs. The TMRE red fluorescence was determined (10,000 cells) on a Becton Dickinson FacsAria flow cytometer. Although data shown are with TMRE, near identical results were obtained with JC-1. It should be pointed out that the rapid and transient release of Ca²⁺ from intracellular stores that occurs within seconds of nsPEF applications has already occurred when measurements of Ca²⁺ are made in these experiments.

b) Results and Discussion

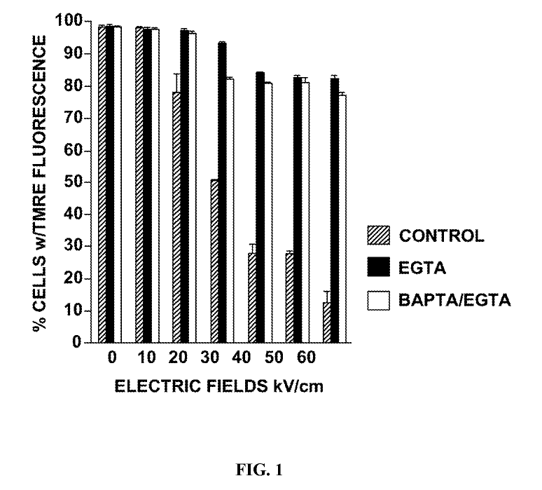

FIG. 1 is a graphical representation illustrating effects of EGTA and EGTA/BAPTA on nanosecond pulsed electric field (nsPEF)-induced dissipation of mitochondria membrane potential (ΔΨm) in Jurkat A3 cells, according to an embodiment.

Jurkat cells (A3 clone) were pre-incubated with TMRE (200 nM) for 20 min. Cells were incubated for 30 min in the presence or absence of BAPTA-AM (20 μM) and/or EGTA (5 mM) and then treated with ten 60 ns pulses (5 ns rise-fall time, 1 Hz) at various electric field strengths up to 60 kV/cm. Cell were then analyzed by flow cytometry 10 min after treatment for red (TMRE, ΔΨm) fluorescent and expressed as percent cells showing fluorescence (Y-axis) at each electric field strength (X-axis). In the presence of 3 mM Ca²⁺ (absence of EGTA) there was an electric field-dependent decrease in cells expressing a high ΔΨm as indicated by decreases in TMRE fluorescence. Near identical results were observed using JC-1 as the ΔΨm indicator (data not shown). There were no differences when Ca²⁺ was chelated from the extracellular environment with EGTA or when chelated from the extracellular and intracellular environment with EGTA and BAPTA-AM. BAPTA-AM alone was not sufficient to chelate influxes of Ca²⁺ from the extracellular environment (data not shown). Decreases in ΔΨm were statistically significant at electric fields≧20 kV/cm. Inhibition by EGTA and BABTA/EGTA were statistically significantly different than control at electric fields≧20 kV/cm. For electric fields greater than 30 kV/cm values for conditions in the presence of EGTA and BABTA/EGTA were statistically significantly different than control. Statistical significance was determined by the paired Student's t-test (p<0.05; n=3). All values indicate mean±SEM. At 60 kV/cm nearly 90% of cells exhibited a low ΔΨm. At all electric fields, the presence of EGTA or EGTA/BAPTA prevented losses in ΔΨm, such that at 60 kV/cm about 80% of cells exhibited Ca²⁺ dependence for nsPEF-induced dissipation of ΔΨm. However, at an electric field≧40 kV/cm neither EGTA nor EGTA/BAPTA completely blocked loss of ΔΨm, thereby indicating that there was also a Ca²⁺-independent effect on ΔΨm. Thus, it can be concluded that there are two thresholds for effects of nsPEFs for loss of ΔΨm: A lower threshold event is Ca²⁺-dependent and a higher threshold event is Ca²⁺-independent. The same conclusions were reached using rat N1-S1 hepatocellular carcinoma cells (data not shown).

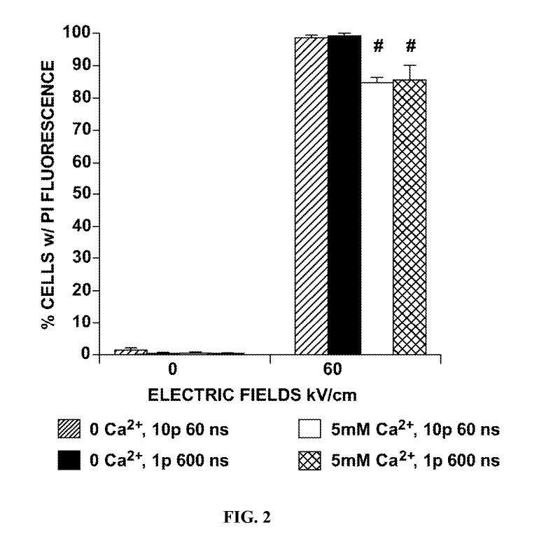

FIG. 2 is a graphical representation illustrating effects of Ca²⁺ on nsPEF-induced permeabilization of plasma membranes, according to an embodiment.

The loss of ΔΨm was dependent on the presence of Ca²⁺ for electric fields that caused ≧60% cell death indicating that poration of the inner mitochondrial membrane (IMM) was not involved; because poration of plasma membranes does not require Ca²⁺. To confirm that Ca²⁺ had no effect on plasma membrane poration, Jurkat A3 cells were treated with one 600 ns pulse with a 10 ns rise-fall time or ten 60 ns pulses with electric field strengths of 0 (sham) or 60 kV/cm, and evaluated propidium iodide (PI) uptake in the presence of 5 mM EGTA (0 Ca²⁺) or 5 mM added Ca²⁺. Cells were then analyzed by flow cytometry ten (10) minutes after treatment for PI fluorescent and expressed as percent cells showing fluorescence (Y-axis). The symbol (#) indicates values were statistically significantly different than their corresponding 0 Ca²⁺ condition as determined by paired Student's t-test (p<0.05; n=3). All values indicate mean±SEM. Control, non-pulsed cells exhibited no PI uptake (data not shown). In the presence and absence of Ca²⁺, significant numbers of cells were porated—taking up PI. While the differences were small, a slightly lower percentage of cells were porated when pulsed in the presence of Ca²⁺. This is due to a role of Ca²⁺ in membrane repair between the time of pulsing and analysis by flow cytometry (10 min). Therefore, a lesser effect on ΔΨm with some repair of the inner mitochondria membrane may occur during a poration event. In other words, the small differences associated with the decrease in PI uptake in the presence of Ca²⁺ could not account for a greater drop in ΔΨm in the presence of Ca²⁺ as a membrane permeabilization related incident. Physical principles indicate that plasma membrane poration events would also hold for poration of intracellular membranes including the inner mitochondria membrane. Thus, the Ca²⁺ dependent drop on ΔΨm is not due to poration of the IMM.

From results in FIGS. 1-2, it can be concluded that there are two thresholds for effects of nsPEFs for loss of ΔΨm. A lower threshold event is Ca²⁺-dependent, is predominant in significant populations of cells, and is unrelated to poration of inner mitochondria membranes. A second threshold is Ca²⁺-independent, requires higher electric fields, and is likely due to permeabilization of inner mitochondria membranes. Given that essentially all Ca²⁺ effects are mediated by proteins, the predominant nsPEF-induced loss of ΔΨm is due to effects on proteins in addition to poration of the inner mitochondria membrane at higher electric fields. Since high frequency components of nsPEFs had greater effects in dissipating ΔΨm, the effects on proteins are also due to high frequency components of nsPEFs. Based on the aforementioned results, nsPEFs can be used for altering intracellular structures and functions of proteins. In light of the Ca²⁺-dependent events illustrated herein on ΔΨm as a poration-independent event, Ca²⁺-independent dissipation of ΔΨm enables permeabilization of the inner mitochondria membrane. While Ca²⁺-dependent dissipation of ΔΨm enabling effects on a protein(s) is not direct, but is by association, it directs attention towards other possible effects of nsPEFs besides permeabilization of lipid membranes.

### Example 2. Inhibitors of the Mitochondria Permeability Transition Pore (mPTP) Complex on nsPEF-Induced Loss of ΔΨm

a) Candidates for nsPEF Targets in the Mitochondria

The protein candidates for nsPEF-induced loss of ΔΨm are in the mitochondria permeability transition pore (mPTP) complex and/or proteins that reside nearby it or associate with it. Various molecular components in the IMM and outer mitochondria membrane (OMM) as well as other interacting molecules have been considered part of mPTP, which is a large, non-selective entity allowing passage of molecules as large as 1.5 kDa across mitochondrial membranes, thereby resulting in organelle swelling and eventual rupture. The mPTP equates to a pore with an open diameter of about 2.0-2.6 nm allowing passage of metabolites as well as hydrated inorganic ions, including Ca²⁺. A prototypic mPTP complex is composed of the voltage-gated anion channel (VDAC) in the OMM, the anion nucleotide transporter (ANT) and the mitochondrial phosphate carrier (PiC) in the IMM and Ca²⁺-dependent cyclophilin D (CypD), which acts as a physiological regulator of mPTP. However, more recently VDAC and ANT have been shown to be dispensable for mPTP activation. In contrast, CypD knock-out mice exhibit resistance to activation of mPTP and to cell death, demonstrating an essential role for this channel and suggests importance of CypD in its regulation. More recently, it has been discovered that the J-protein DnaJC15, which is known to transport precursor into organelles such as mitochondria, recruits and couples CypD with mitochondria permeability transition. Further, elevated DnaJC15 in association with CypD have caused mPTP activation, elevated Ca²⁺, and loss of ΔΨm. The Ca²⁺-dependent loss of ΔΨm and cell death in response to nsPEFs is consistent with the two-hit hypothesis of cell injury and death resulting from elevated intracellular Ca²⁺ and specific damage to mitochondria. In the case of nsPEFs, the elevated intracellular Ca²⁺ and specific damage to mitochondria are due to the influx of Ca²⁺ after plasma membrane poration and damage to mitochondria. Specifically, the elevated intracellular Ca²⁺ and specific damage to mitochondria are due to nsPEF-induced effects on some protein module(s) or protein—lipid complexes in mitochondrial membranes.

Furthermore, while Ca²⁺ overload is a major feature of cell injury, it alone is innocuous. This is based on findings in heart cells and vascular smooth muscle that >100-fold increase in mitochondrial Ca²⁺ allowed maintenance of cellular ATP and cells maintained viability. These findings are consistent with the above findings in N1-S1 hepatocellular carcinoma cells in that a 600 ns pulse waveform with a slow rise-fall time and a mismatched load resulted in large populations of cells with high levels of intracellular calcium, but these cells did not lose ΔΨm and they remained viable. In some embodiments, opening the mPTP dissipates ΔΨm because ΔΨm is Ca²⁺-dependent and includes a voltage-dependent element. In these embodiments, inhibitors of associated mPTP components provide information about how nsPEF-induces Ca²⁺-dependent losses of ΔΨm.

b) Materials and Methods

N1-S1 cells were incubated with several reagents that affect mPTP. Cyclosporin A (CsA) (5 μM) was incubated for 15 min; RR (5 μM) was incubated for 15 min; DIDS (100 μM) for ten (10) min; BKA (50 μM) for 15 min. Cells were then exposed to various electric fields and then assayed 15 min after pulsing and TMRE fluorescence was determine by flow cytometry, as indicated in Example 1. Cyclosporin was used to inhibit cyclophilin D, bongkrekic acid was used to inhibit the ANT, and DIDS (disodium 4,4′-diisothio-cyanatostilbene-2,2′-disulfonate) was used to inhibit the voltage-dependent ion channel (VDAC).

c) Results and Discussion

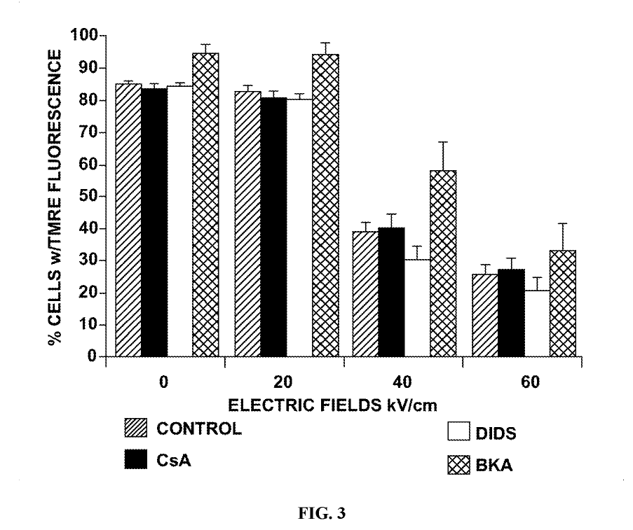

FIG. 3 is a graphical representation illustrating effects of inhibitors of the mitochondria permeability transition pore (mPTP) complex on nsPEF-induced loss of ΔΨm, according to an embodiment.

The ΔΨm was determined in the presence and absence of inhibitors of possible mPTP components. As observed in FIG. 3, none of the inhibitors had significant effects on nsPEF-induced dissipation of ΔΨm. This suggested that the mPTP is not a participant in the nsPEF-induced dissipation of ΔΨm. In addition to nsPEFs effects on cell membranes, nsPEFs may non-transiently and possibly directly affect a protein(s) that modulates the mPTP. In some embodiments, none of the compounds were statistically significantly different than the control compound at each of the corresponding electric fields. In these embodiments, each compound was significantly different from 0 kV/cm control field at 40 kV/cm and 60 kV/cm only. To determine the significance, the paired Student's t-test was used (p<0.05; n=3). In FIG. 3, all values indicate mean±SEM.

### Example 3. Inhibition of the Activity of the cAMP-Dependent Protein Kinase Catalytic-C Subunit

a) Materials and Methods

Expression and Purification of the PKA Cα-Subunit

Recombinant murine his₁₀-Cα was expressed overnight from a pET16b expression vector from IPTG (about 0.4 mM) induced BL231(DE3)pLysS (Novagen) transformed competent cells in the presence of ampicillin (about 50 μg/mL) at about 37° C. The C-subunit was purified by a variation of the method such as, for example as described by Zhang et al. (W. Zhang, G. Z. Morris, S. J. Beebe, Characterization of the cAMP-dependent protein kinase catalytic subunit Cgamma expressed and purified from sf9 cells, Protein Expr. Purif. 35 (2004) 156-169). The induced bacterial suspension was centrifuged at about 11,500 g at about 4° C. for about 2 h. The pelleted cells were sonicated in binding buffer (about 50 mM NaPO₄, pH 7.9, 0.5 M NaCl, and 10% glycerol) with about 2 mM PMSF. The extract was loaded onto a Ni-IMAC column (Probond, Invitrogen) and washed with binding buffer with about 60 mM imidazole. The Ca-subunit was eluted with a gradient of about 0.06-1.0 M imidazole followed by separation on a Sephadex 5300 gel filtration column equilibrated in about 50 mM NaPO₄, pH 6.9, 150 mM NaCl. The homogeneous enzyme was stable for several weeks when stored at about 4° C. without loss of activity.

The enzyme was suspended in Hanks balance salt solution with Ca²⁺ and exposed to one or ten pulses with durations of either 60 ns and 60 kV/cm or 300 ns and 26 kV/cm. Fifteen minutes after pulsing the kinase as assayed for catalytic activity as previously described above.

NsPEF Treatment and Assay of PKA C-Subunit Activity

Recombinant C-subunit in about 50 mM NaPO₄, pH 6.9, 150 mM NaCl was treated with nsPEFs in about 1 mm cuvettes in the same way that cell suspensions are exposed to nsPEFs, such as, for example as described by Schoenbach et al. (K. H. Schoenbach, S. J. Beebe, E. S. Buescher, Intracellular effect of ultrashort electrical pulses, Bioelectromagnetics 22 (2001) 440-448). Twenty (20) minutes after treatment, Ca-subunit activity was determined by the transfer of [γ-³²P]ATP (about 200 μM) to peptide (about 65 μM Kemptide, Leu-Arg-Arg-Ala-Ser-Leu-Gly), such as, for example as described by Zhang et al. (see above) using the filter paper assay, such as, for example as described by Roskowski (R. Roskowski, Assays of protein kinase, Methods Enzymol. 99 (1983) 3). C-subunit activity is expressed in ³²P-incorporation into peptide as counts per minute.

b) Results and Discussion

In vitro experiments were performed to determine whether or not nsPEFs have direct effects on enzyme activity of the catalytic subunit (C-subunit) of the cAMP-dependent protein kinase (PKA). Protein kinases (PKAs) are ubiquitous enzymes that regulate diverse cell functions by transferring the γ-phosphate from ATP to serine, threonine or tyrosine residues in substrate proteins. This leads to conformational changes within the phosphorylated protein, altering its function. Thus, phosphorylation/dephosphorylation is one of the mechanisms within the cell that regulates an extensive range of physiological and pathological functions. Because all kinases contain a highly conserved catalytic core that is essential for catalysis, they share common structures and catalytic mechanisms of action. The PKA C-subunit (350 amino acids) is the simplest of the protein kinase super family and therefore can be used as a prototype for any member of the protein kinase super family. The C-subunit is well-defined by its crystal structure. The C-subunit comprises a smaller N-terminal lobe (amino acids 40-119), which is dominated by anti-parallel beta-sheets, that binds and orients ATP and a larger C-terminal lobe (amino acids 128-300), which includes alpha-helices, that binds the substrate and transfers phosphate from ATP to the substrate. A small linker sequence (amino acids 120-127) connecting the two lobes, aids in substrate recognition, and anchors the ATP/Mg. A C-terminal tail (amino acids 301-350) wraps over the entire core structure. The catalytic site resides between the two lobes. Like all proteins, the function of the PKA C-subunit is determined by its specific active structure.

C-subunit regulation is fundamentally important for a wide range of biological processes, such as, for example memory, metabolism, growth development and apoptosis. Within the context of apoptosis, the PKA-C-subunit plays an important role in cell survival based on its localized function at the OMNI. However, to understand the importance of the PKA C-subunit in maintenance and survival functions, other subunits and proteins need to be considered. The PKA holoenzyme is composed of a regulatory (R) subunit dimer that binds cAMP and two C-subunit monomers that are kinases. The PKA holoenzyme family is composed of four different R-subunits (RIα, RIβ, RIIα, RIIβ) and three different C-subunit isozymes (Cα, Cβ, Cγ; the latter is testis- and human-specific). The R-subunit classifies the family as type I or type II PKA. When cAMP binds to the R-subunit, the two C-subunit monomers are released, freeing their catalytic sites to phosphorylate appropriate substrates, thereby modifying their substrate's function. The R-subunits target C-subunits to various subcellular localizations by binding to a family of A kinase anchoring proteins (AKAPs), which are considered as possible therapeutic targets. The first identified AKAP-1 localized predominantly type II PKA to the OMM. Localizing C-subunits to OMNI is important to maintain mitochondrial function and survival. Point mutations in AKAP-1 that prevented PKA binding increased the sensitivity of PC12 cells to apoptotic stimuli. Overexpression of AKAP-1 mitigated apoptosis induced by serum starvation and resulted in phosphorylation and inhibition of the BH-3-only pro-apoptotic protein as well as Bcl-2-associated death promoter (BAD). When BAD is not phosphorylated, it heterodimerizes with BCL-2 or BCL-Xl at OMM sites to promote cell death. When phosphorylated, BAD is sequestered to the cytosol by binding the 14-3-3 protein thereby allowing BCL-2 and/or BCL-XL to promote survival. In some embodiments, displacement of AKAP-1 from the OMM with an AKAP-1 peptide reduces oxidative ATP synthesis, decreases ΔΨm and increases oxidative stress thereby resulting in cardiomyocyte death. In these embodiments, IL-3 survival signals initiating activation of PKA that is targeted to the OMM where BAD is phosphorylated and inactivated. Further to these embodiments, AKAP-1-mediated localization of PKA to mitochondria is a natural strategy to locate a C-subunit near its substrate BAD and protect mitochondria in the presence of IL-3.

The PKA C-subunit is also involved in maintenance of mitochondrial morphology, which is important for ATP production, mitochondrial transport and apoptosis. Mitochondria are dynamic organelles whose shape is determined by fission and fusion reactions catalyzed by G-proteins in the dynamin family. Generally, fission reactions are induced by phosphatases while fusion reactions or reactions that cause unopposed fusion are determined by kinases, including PKA. The C-subunit phosphorylates dynamin-related protein 1 (Drp-1), which is a large GTPase that physically restricts and severs mitochondria. AKAP-1 increases the localization of C-subunit at the OMNI thereby allowing phosphorylation of Ser-637 resulting in Drp-1 inhibition. In hippocampal neurons, AKAP-1 knockdown causes mitochondrial fragmentation and apoptosis, while overexpression of AKAP-1 and phosphorylation of Drp-1 on Ser-637 are neuroprotective by promoting mitochondrial elongation and unopposed fusion. The role of PKA anchoring and maintenance of integrity (elongation) and survival is played out when AKAP-1-anchored PKA-C-subunit phosphorylates Drp-1, which inhibits its disassembly step in the catalytic cycle, accumulating large, slowly recycling Drp-1 oligomers reducing their fission functions. These conditions of unopposed fusion promote formation of mitochondria reticulum and elongation, thereby promoting their survival function.

Most AKAPs bind to any RII-subunit (type II PKA) with a high affinity, but sphingosine kinase interacting proteins (SKIPs) specifically bind to the type I PKA with the highest affinity. SKIPs are localized to the peripheral inner mitochondrial membrane space where they are associated with an important C-subunit substrate, the coiled coil helix protein ChChd3. ChChd3 is crucial for maintaining crista integrity and mitochondrial function. RNAi knockdown of ChChd3 in HeLa cells results in fragmented mitochondria, impaired fusion, clustering of mitochondria around the nucleus, severely restricted oxygen consumption and glycolytic rates and reduced growth rates. Ultrastructural analysis of these cells revealed aberrant mitochondrial inner membrane structures. Other data indicate that ChChd3 is a scaffolding protein that stabilizes protein complexes with other proteins involved in maintaining crista architecture. This data indicates that PKA type I C-subunit phosphorylation activity plays a role in mitochondrial integrity from a different perspective than PKA type II C-subunit plays, through phosphorylation and regulation of Drp-1 and for a different survival function than that served by phosphorylation of BAD.

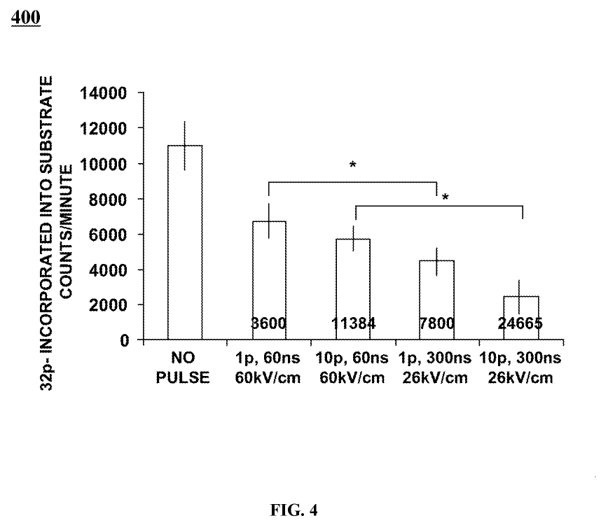

The data provides substantial evidence that AKAP-mediated localization of PKA C-subunit to mitochondria and phosphorylation of several regulatory proteins are important for survival functions and mitochondria integrity in several cell types, including cardiomyocytes and neurons. The absence of AKAP-mediated mitochondrial localization and the corresponding loss of C-subunit phosphotransferase activity near and/or in mitochondria induced a plethora of functional maladies including, among others, loss of ΔΨm, which was observed in nsPEF treated Jurkat cells. Given the importance of C-subunit activity for mitochondrial function and survival and possible effects of nsPEFs on proteins, it was determined nsPEF directly affects the catalytic activity of this prototypic protein kinase (see FIG. 4 discussion).

FIG. 4 is a graphical representation illustrating effects of nsPEFs on enzyme activity of the catalytic subunit of the cAMP-dependent protein kinase (PKA), according to an embodiment.

In this study, a mouse recombinant Cα-subunit containing a His-tag (for ease of purification) was expressed in E. coli and purified to homogeneity on a nickel column as previously described above. To determine if nsPEF could affect the structure of PKA C-subunit, the recombinant protein was treated with nsPEFs and then its catalytic activity was determined by ³²P incorporation into a peptide substrate (Leu-Arg-Arg-Ala-Ser-Leu-Gly, Kemptide) as a specific measure of its function (see FIG. 4). The treatments were applied with one or ten (10) pulses at 60 ns and 60 kV/cm and with one or ten (10) pulses at 300 ns and 26 kV/cm. Additionally, ten (10) pulses were delivered at 0.5 Hz (1 pulse/2 s). For each of the one and ten pulse conditions, the energy densities are equivalent and there were no increases in temperature. NsPEFs caused a 41% and 45% loss in activity for one and ten pulses at 60 ns, respectively. Further, nsPEFs caused a 55% and 77% loss for one and ten pulses at 300 ns, respectively. Given that the one pulse and ten pulse conditions between 60 ns, 60 kV/cm and 300 ns, 26 kV/cm exhibited similar energy densities, the inhibitory effects on kinase structure/function appear to be, at least in part, energy-independent. The same relationships have also been shown for effects on nsPEF on ethidium homodimer uptake across plasma membranes and on activation of caspase. When these results are considered regarding the scaling factor that reflects the charge transferred through the kinase solution using the formula Eτn^{0.5}, for a given pulse number, greater effects were observed at the 300 ns and 26 kV/cm condition when compared to the 60 ns and 60 kV/cm conditions. This can be seen by the numbers in the bars in FIG. 4, which represent the charge transferred to the kinase by the above formula. Thus, the charging factor accounts for differences seen for conditions with the same pulse numbers that have the same energy density. Since the functional catalytic activity of the C-subunit is dependent on its active structure, nsPEFs caused a structural change or an unfolding response in the kinase, resulting in its inactivation. Furthermore, displacement of the C-subunit from OMNI resulted in decreased ΔΨm and cardiomyocyte death.

The nsPEF induces a loss of secondary structure that is critical for catalytic activity in significant populations of C-subunit proteins. Considering the structural features of the kinase, nsPEFs disrupt H-bonding within the catalytic cleft formed with the closure of the two lobes upon catalysis. These new H-bonds are formed between two amino acids in the ATP-binding site in the small lobe; one amino acid in the catalytic loop and three residues in the Kemptide substrate. The nsPEFs interfere with hydrogen bonding, but the bonds can be reestablished making them temporary due to the time between the pulse exposure and the kinase assay (20 min). Therefore, disrupting hydrophobic interactions would also seem to be recoverable. It would be even less likely that nsPEFs could disrupt the H-bonds that establish the stability within the small and large lobes. Considering that nsPEF would affect H-bonding or hydrophobic interactions would most likely address whether enough energy was deposited into the structure to cause such changes. Another factor to consider is that kinase inhibition is due to high frequency components of nsPEFs. Pulsing with high frequency components compared to pulsing without these components were more effective to disrupt the ΔΨm and that this loss of ΔΨm was Ca²⁺-dependent, which is not consistent with a poration event. This is because nsPEFs with high frequency components have direct effects on protein(s) as indicated by the kinase data.

In summary, nsPEFs have effects on proteins in addition to permeabilizing cellular membranes. The findings that dissipation of ΔΨm is Ca²⁺-dependent means that this was not due to poration of the inner mitochondria membrane. It was shown that the presence of high Ca²⁺ slightly inhibited permeabilization of the plasma membrane. Based on physical principles, this same finding will hold for intracellular membranes, including the inner mitochondria membrane. Since essentially all Ca²⁺-dependent events require proteins, nsPEFs act on a protein(s) to account for dissipation of ΔΨm. Additionally, nsPEFs inactivate the C-subunit of PKA, a prototype for all protein kinases, which have highly conserved catalytic mechanisms. As observed for other nsPEF effects, this inactivation is independent of energy density and related to a charging effect defined by the formula Eτn^{0.5}. Given that nsPEF-induced dissipation of ΔΨm was more effective when high frequency components were present in fast rise time waveforms, it is possible that effects on proteins are due to these high frequency components.

Since the basic catalytic mechanisms for phosphorylation in all kinases are highly conserved, nsPEFs affects one of the largest protein families, which are involved in essentially all aspects of cell function and account for 2% of the mammalian genome, 47% of which map to disease loci or cancer amplicons. Further, lipid-protein pore complexes embedded in cell membranes are affected by nsPEFs to account for other observed effects that are now attributed to effects on cell lipid membranes only.

While various embodiments of the present disclosure have been described above, it should be understood that they have been presented by way of example only, and not limitation. Numerous changes to the disclosed embodiments can be made in accordance with the disclosure herein without departing from the spirit or scope of the disclosure. Thus, the breadth and scope of the present disclosure should not be limited by any of the above described embodiments.

Rather, the scope of the disclosure should be defined in accordance with the following claims and their equivalents.

Although the present disclosure has been illustrated and described with respect to one or more implementations, equivalent alterations and modifications can occur to others skilled in the art upon the reading and understanding of this specification and the drawings. In addition, while a particular feature of the disclosure may have been disclosed with respect to only one of several implementations, such feature may be combined with one or more other features of the other implementations as may be desired and advantageous for any given or particular application.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

What is claimed is:

1. Method of manipulating cellular function of a cell, comprising:
providing at least one cell having an intracellular protein; and
applying at least one nanosecond pulsed electric field to the at least one cell, the electric field having high frequency components present in fast rise time waveforms, at a strength and for a duration sufficient to alter the structure of the intracellular protein of the at least one cell.

2. The method of claim 1, wherein the cellular function of the at least one cell is altered due to the altered structure of the intracellular protein.

3. The method of claim 1, wherein the at least one cell comprises one of a prokaryotic cell or eukaryotic cell.

4. The method of claim 1, wherein the at least one cell comprises a Jurkat cell.

5. The method of claim 1, wherein the at least one cell comprises a fat cell, bone cell, vascular cell, muscle cell, cartilage cell, or stem cell, or a combination thereof.

6. The method of claim 1, wherein the at least one cell is abnormal.

7. The method of claim 1, wherein the at least one cell is a cancer cell.

8. The method of claim 1, wherein the electric field has a pulse duration of at least about 60 nanoseconds.

9. The method of claim 1, wherein the electric field has a pulse duration of no more than about 600 nanoseconds.

10. The method of claim 1, wherein the electric field has a strength from about 0 kV/cm to about 60 kV/cm.

11. The method of claim 1, wherein the intracellular protein comprises a protein embedded in a plasma membrane.

12. The method of claim 11, wherein the plasma membrane is mitochondrial membrane.

13. The method of claim 1, wherein the intracellular protein is part of a protein-lipid complex.

14. The method of claim 1, wherein the application of the at least one nanosecond pulsed electric field alters the catalytic activity of the intracellular protein.

15. The method of claim 14, wherein the intracellular protein is a C-subunit of cAMP-dependent protein kinase.
